(19) United States
(12) Patent Application Publication
(10) Pub. No.: US 2025/0093265 A1
(43) Pub. Date: Mar. 20, 2025

(54) OPTICAL SENSOR AND DETECTION DEVICE

(71) Applicant: Murata Manufacturing Co., Ltd., Nagaokakyo-shi (JP)

(72) Inventor: Yasuo FUJII, Nagaokakyo-shi (JP)

(21) Appl. No.: 18/965,135

(22) Filed: Dec. 2, 2024

Related U.S. Application Data
(63) Continuation of application No. PCT/JP2023/019705, filed on May 26, 2023.

(30) Foreign Application Priority Data
Jun. 10, 2022 (JP) 2022-094448

(51) Int. Cl. G01N 21/64

(52) U.S. Cl. CPC G01N 21/6428; G01N 2021/6432; G01N 2021/6439

(57) ABSTRACT

An optical sensor that includes a fluorescent unit containing a plurality of kinds of quantum dots, enzymes, and quenchers in a hydrogel.

### CROSS REFERENCE TO RELATED APPLICATIONS

The present application is a continuation of International application No. PCT/JP2023/019705, filed May 26, 2023, which claims priority to Japanese Patent Application No. 2022-094448, filed Jun. 10, 2022, the entire contents of each of which are incorporated herein by reference.

### TECHNICAL FIELD

The present disclosure relates to an optical sensor and a detection device.

### BACKGROUND ART

Non-Patent Document 1 discloses a biosensor using a quantum dot and an enzyme. In the biosensor described in Non-Patent Document 1, quantum dot-enzyme conjugates are confined in a hydrogel.

Non-Patent Document 1: Biosensors and Bioelectronics 31 (2012) 529-536

### SUMMARY OF THE DISCLOSURE

In the example of Non-Patent Document 1, a biosensor is configured by arranging a glucose sensor using glucose oxidase (GOX) as an enzyme and an alcohol sensor using alcohol oxidase (AOX) as an enzyme in the middle of a microchannel.

However, the biosensor described in Non-Patent Document 1 can detect only one of glucose and alcohol using one sensor. Accordingly, it is desired to detect a plurality of items using one sensor.

An object of the present disclosure is to provide an optical sensor capable of accurately detecting a plurality of items. Furthermore, an object of the present disclosure is to provide a detection device including the optical sensor.

An optical sensor of the present disclosure includes a fluorescent unit containing a plurality of kinds of quantum dots, enzymes, and quenchers in a hydrogel.

A detection device of the present disclosure includes: a container having optical transparency; a sensor unit arranged inside the container; and a light emitting element and a light receiving element arranged outside the container so as to face the sensor unit, in which the sensor unit is the optical sensor of the present disclosure.

According to the present disclosure, an optical sensor capable of accurately detecting a plurality of items can be provided. Furthermore, according to the present disclosure, a detection device including the optical sensor can be provided.

### BRIEF EXPLANATION OF THE DRAWINGS

FIG. 1 is a schematic view for explaining a light emission principle of a quantum dot.

FIG. 2 is a schematic view for explaining a quenching principle of a quantum dot by a quencher.

FIG. 3 is a schematic view for explaining an example of combinations of a quantum dot and a quencher.

FIG. 4 is a schematic view for explaining another example of combinations of a quantum dot and a quencher.

FIG. 5 is a schematic view illustrating an optical sensor according to an embodiment of the present disclosure.

FIG. 6A is a schematic view illustrating an example of a red quantum dot contained in a hydrogel. FIG. 6B is a schematic view illustrating an example of a green quantum dot contained in a hydrogel.

FIG. 7 is a graph illustrating the relationship between the glucose concentration and the lactic acid concentration, and the green fluorescence peak intensity.

FIG. 8 is a graph illustrating the relationship between the glucose concentration and the lactic acid concentration, and the red fluorescence peak intensity.

FIG. 9 is a schematic view illustrating an example of a state in which a quantum dot and enzymes are conjugated.

FIG. 10 is a schematic view illustrating a detection device according to an embodiment of the present disclosure.

FIG. 11 is a schematic view in which a part of the detection device illustrated in FIG. 10 is enlarged.

FIG. 12 is a schematic view illustrating a detection device according to another embodiment of the present disclosure.

### DESCRIPTION OF THE PREFERRED EMBODIMENTS

Hereinafter, a description is made of an optical sensor and a detection device of the present disclosure. The present disclosure is not limited to the following configuration, and may be modified as appropriate without changing the gist of the present disclosure. The present disclosure also includes a combination of a plurality of individual preferable configurations described below.

The following drawings are schematic views, and the dimensions, the scales of aspect ratios, and the like may be different from those of actual products.

### [Optical Sensor]

An optical sensor of the present disclosure includes a fluorescent unit containing a plurality of kinds of quantum dots, enzymes, and quenchers in a hydrogel.

The optical sensor of the present disclosure utilizes an enzyme reaction that causes an enzyme to react with a detection target. In this method, various substances can be detected by changing the enzyme.

Furthermore, the optical sensor of the present disclosure utilizes a phenomenon in which electrons excited in the quantum dot transfer to a quencher. As described later, the quencher also includes a quencher generated by an enzymatic reaction (H₂O₂ or the like). In addition, a coenzyme that assists the action of an enzyme may function as the quencher.

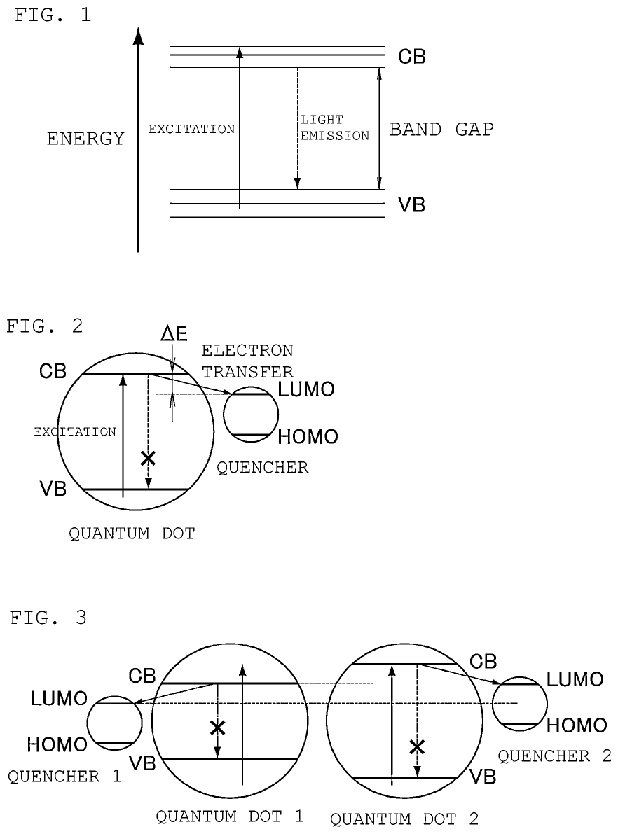

FIG. 1 is a schematic view for explaining a light emission principle of a quantum dot.

As illustrated in FIG. 1, when electrons in the quantum dot are excited, fluorescence is emitted at the time when the electrons in the excited state return to the ground state. Specifically, when electrons in the valence band VB are excited to the conduction band CB by excitation light, and the electrons return from the conduction band CB to the valence band VB, fluorescence is emitted. The band gap illustrated in FIG. 1 corresponds to the color (emission wavelength) of the quantum dot. When there is a defect level below the conduction band CB, fluorescence may be emitted at the time when the electrons return from the defect level to the valence band VB.

FIG. 2 is a schematic view for explaining a quenching principle of a quantum dot by a quencher.

As illustrated in FIG. 2, when electrons excited to the conduction band CB transfer from the quantum dot to a quencher, the quantum dot does not emit fluorescence. In order for electrons to transfer, it is considered that there is an optimum value for the energy difference ΔE between a quantum dot and a quencher (Markus theory). In FIG. 2, LUMO represents the lowest unoccupied molecular orbital, and HOMO represents the highest occupied molecular orbital. FIG. 2 illustrates an example in which electrons transfer to LUMO, but electron transfer to another molecular orbital may also be utilized. The same applies to FIGS. 3 and 4 described later.

In the optical sensor of the present disclosure, a plurality of kinds of quantum dot, enzyme, and quencher are confined in a hydrogel. As a result, a plurality of items can be detected using one sensor. Conversely, when the quencher reacts with the plurality of kinds of quantum dot, the detection accuracy may be lowered.

However, the quantum dots used as phosphors have small wavelength dispersions, and the center wavelength can be adjusted by size, composition, and the like, so that wavelength bands of a plurality of colors can be prevented from overlapping with each other for the respective quantum dots. In this way, in the optical sensor of the present disclosure, since color adjustment is facilitated by using the quantum dots as phosphors, a color can be selected in which electrons easily transfer to the quencher. Accordingly, the quencher can be suppressed from reacting with the plurality of kinds of quantum dot, so that a plurality of items can be detected with high accuracy.

As long as at least one quencher reacts only with one quantum dot, when another quencher reacts with a plurality of quantum dots, both peaks can be separated from each other, so that respective items can be detected with high accuracy. Accordingly, in the optical sensor of the present disclosure, it is sufficient that at least one quencher reacts only with one quantum dot, but it is preferable that all quenchers react with a plurality of kinds of quantum dot in a one-to-one relationship.

FIG. 3 is a schematic view for explaining an example of combinations of a quantum dot and a quencher.

As shown in FIG. 3, as a combination of a certain kind of quantum dot and a quencher that quenches fluorescence by the quantum dot, it is preferable to combine a quencher having a low LUMO energy level (quencher 1 in FIG. 3) with a quantum dot having a low energy level at the lower end of the conduction band CB (quantum dot 1 in FIG. 3), and combine a quencher having a high LUMO energy level (quencher 2 in FIG. 3) with a quantum dot having a high energy level at the lower end of the conduction band CB (quantum dot 2 in FIG. 3). The same applies to the combination of a quantum dot and a quencher in the case of three or more kinds.

Alternatively, as a combination of a certain kind of quantum dot and a quencher that quenches fluorescence by the quantum dot, it is preferable to combine a quencher having a low LUMO energy level (quencher 1 in FIG. 3) with a quantum dot having a small band gap (quantum dot 1 in FIG. 3), and combine a quencher having a high LUMO energy level (quencher 2 in FIG. 3) with a quantum dot having a large band gap (quantum dot 2 in FIG. 3). The same applies to the combination of a quantum dot and a quencher in the case of three or more kinds.

FIG. 4 is a schematic view for explaining another example of combinations of a quantum dot and a quencher.

As shown in FIG. 4, as a combination of a certain kind of quantum dot and a quencher that quenches fluorescence by the quantum dot, a quencher having a high LUMO energy level (quencher 1 in FIG. 4) may be combined with a quantum dot having a high energy level at the lower end of the conduction band CB (quantum dot 1 in FIG. 4), and a quencher having a low LUMO energy level (quencher 2 in FIG. 4) may be combined with a quantum dot having a low energy level at the lower end of the conduction band CB (quantum dot 2 in FIG. 4). Unlike the example illustrated in FIG. 3, in the example illustrated in FIG. 4, a quencher having a high LUMO energy level (quencher 1 in FIG. 4) is combined with a quantum dot having a small band gap (quantum dot 1 in FIG. 4), and a quencher having a low LUMO energy level (quencher 2 in FIG. 4) is combined with a quantum dot having a large band gap (quantum dot 2 in FIG. 4). When materials configuring quantum dots are different from each other, the positions of the energy bands are different from each other, so that when the quantum dot 1 and the quantum dot 2 of different materials are used, such a combination of quenchers can be adopted. The same applies to the combination of a quantum dot and a quencher in the case of three or more kinds.

Since the energy level of a quencher is related to the oxidation-reduction potential, the energy level can be measured by electrochemical measurement.

For example, a working electrode (platinum or the like), a reference electrode (silver/silver chloride or the like), and a counter electrode (platinum or the like) are placed in a solution containing a detection target (glucose or the like), and cyclic voltammetry measurement is performed with a potentiostat. On the working electrode, a hydrogel containing an enzyme and a quencher (unnecessary in the case of being generated by an enzymatic reaction like H₂O₂) is formed, and an oxidation-reduction reaction of the quencher occurs in conjunction with an enzymatic reaction. When the current is measured while the voltage is swept, a current peak is observed at the oxidation-reduction potential of a quencher. The energy level of a quencher can be determined from the level of the oxidation-reduction potential.

On the other hand, the energy level of a quantum dot can be measured by a method such as photoelectron spectroscopy or light absorption spectroscopy. In particular, in the case of quantum dots of the same material different in composition ratio (ZAIS and the like described later), as in the example illustrated in FIG. 3, the energy level can be determined also from the magnitude of the band gap (short-wavelength light emission: large band gap, long-wavelength light emission: small band gap).

In the optical sensor of the present disclosure, examples of the quantum dot include a ZnS—AgInS₂ solid solution (ZAIS). By changing the composition ratio of ZAIS, a plurality of kinds of quantum dot having different bandgaps can be prepared. In addition, by using ZAIS as quantum dots, toxicity can be reduced as compared with a quantum dot containing an element such as Cd.

The optical sensor of the present disclosure may further contain a quantum dot for reference in a hydrogel. The reference quantum dot is a quantum dot that hardly reacts with a detection target, and emits constant light as a reference. The reference quantum dot is confined in a protective film such as glass beads, for example.

In the optical sensor of the present disclosure, the quencher may be added into a hydrogel in advance or may be generated by an enzymatic reaction. For example, H₂O₂ serving as the quencher is generated from dissolved O₂. On the other hand, when the quencher is added, the presence or absence of the quencher is not affected by the dissolved O₂ concentration, so that options of enzymes are expanded.

In the optical sensor of the present disclosure, the hydrogel is preferably nonionic. As long as a nonionic hydrogel is used, when a quencher having a charge is used, the quencher is not captured by the charge of the hydrogel, so that the quencher can freely move to quench the quantum dot. Accordingly, options of the quencher are expanded.

The optical sensor of the present disclosure may include a fluorescent layer as the fluorescent unit.

The optical sensor of the present disclosure preferably further includes a support member having optical transparency on a first principal surface side of the fluorescent layer. The fluorescent layer can be supported by the support member.

The support member may be made of an inorganic material or an organic material. Examples of the support member made of an inorganic material include a glass substrate. Examples of the support member made of an organic material include a resin film such as a polyethylene terephthalate (PET) film.

The optical sensor of the present disclosure preferably further includes a light shielding layer on a second principal surface side of the fluorescent layer. In this case, the optical sensor of the present disclosure may further include a reflection layer between the fluorescent layer and the light shielding layer.

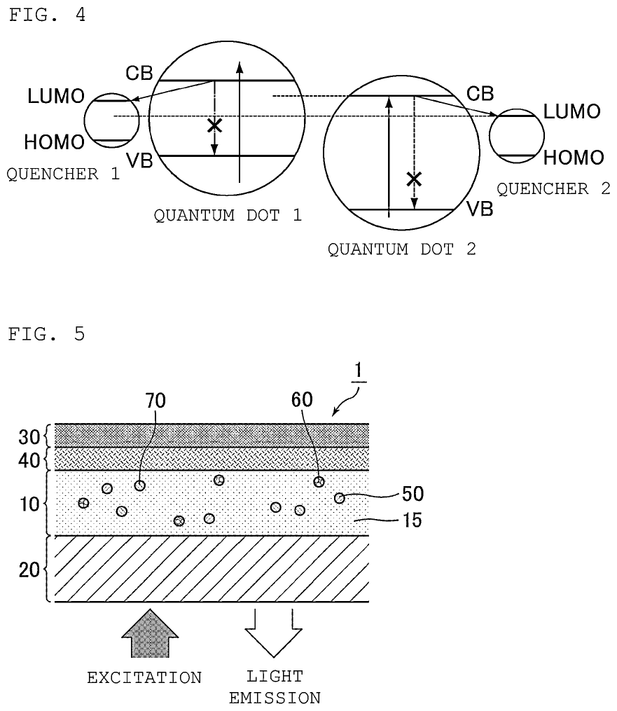

FIG. 5 is a schematic view illustrating an optical sensor according to an embodiment of the present disclosure.

The optical sensor 1 illustrated in FIG. 5 includes the fluorescent layer 10 as the fluorescent unit. The support member 20 is provided on a first principal surface side of the fluorescent layer 10, and the light shielding layer 30 is provided on a second principal surface side of the fluorescent layer 10 opposite the first principal surface side. Furthermore, the reflection layer 40 is provided between the fluorescent layer 10 and the light shielding layer 30.

For example, a PET film is used as the support member 20, and the fluorescent layer 10, the reflection layer 40, and the light shielding layer 30 are sequentially applied onto the support member 20 to manufacture the optical sensor 1.

The fluorescent layer 10 contains a plurality of kinds of quantum dots 50, enzymes 60, and quenchers 70 in a hydrogel 15.

In the light shielding layer 30, carbon black is mixed with a hydrogel of polyethylene glycol (PEG).

In the reflection layer 40, TiO₂ particles are mixed in a hydrogel of polyethylene glycol (PEG).

As an example specifically illustrating the optical sensor of the present disclosure, the optical sensor was manufactured by the following method. Note that the present disclosure is not limited only to the example below.

To a support member was added dropwise 0.8 mL of a mixture of acrylamide: N,N′-methylenebisacrylamide=29:1 4 w/v %, red quantum dots ZAIS-QD 0.17 μM, green quantum dots ZAIS-QD 4.7 μM, glucose dehydrogenase (GDH) 44 units/mL, lactate oxidase (LOx) 2.9 units/mL, β-nicotinamide adenine dinucleotide (NAD) 1.8 mM, ammonium peroxodisulfate 0.1 w/v %, N, N,N′, N′-tetramethylethylenediamine 0.04 v/v % in a phosphate buffered saline (PBS) solution, followed by curing by blocking the air.

GDH and LOx are enzymes, and NAD serves as both a quencher and a coenzyme of GDH. Ammonium peroxodisulfate and N, N,N′, N′-tetramethylethylenediamine are polymerization initiators. M means mol/L.

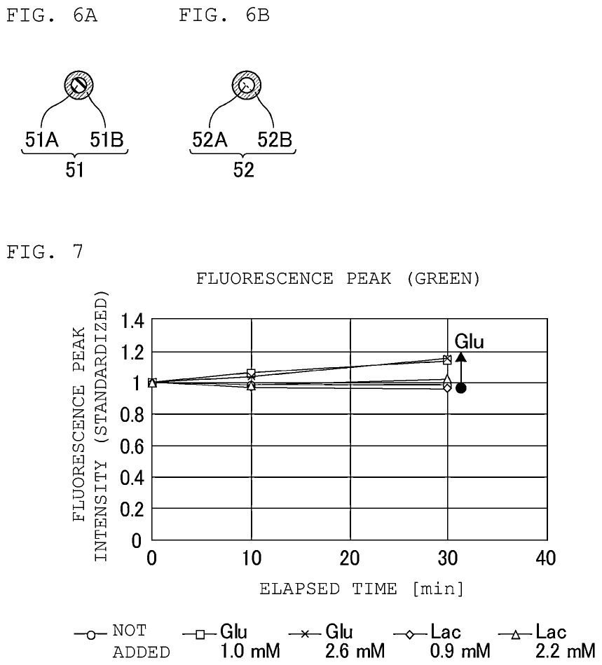

FIG. 6A is a schematic view illustrating an example of a red quantum dot contained in a hydrogel.

The red quantum dot 51 is, for example, a core-shell quantum dot including a core 51A and a shell 51B covering the core 51A.

FIG. 6B is a schematic view illustrating an example of a green quantum dot contained in a hydrogel.

The green quantum dot 52 is, for example, a core-shell quantum dot including a core 52A and a shell 52B covering the core 52A.

Among enzymes contained in the fluorescent layer 10, lactate oxidase (LOx) enzymatically reacts with lactic acid (Lac), which is one of detection targets. As a result, H₂O₂ as a quencher is generated, so that the light emission of the red quantum dot 51 is selectively reduced.

Among enzymes contained in the fluorescent layer 10, glucose dehydrogenase (GDH) enzymatically reacts with glucose (Glu), which is one of detection targets. At the same time, an NAD as a quencher is reduced to make the quenching function disappear, and the light emission of the red quantum dot 51 and the green quantum dot 52 is increased.

For the optical sensor 1 installed in the PBS solution, a fluorescence spectrum was measured using a plate reader.

First, sensor fluorescence was measured by excitation at a wavelength of 450 nm using a plate reader, and a spectrum in which green and red overlap with each other was obtained. Fitting was performed with a Gaussian function for separation into a first peak (green) and a second peak (red). For green and red, the respective peak values were used as measured values.

The relationship between the glucose concentration and the lactic acid concentration, and the green fluorescence peak intensity is shown in Table 1, and the relationship between the glucose concentration and the lactic acid concentration, and the red fluorescence peak intensity is shown in Table 2. The fluorescence peak intensity is a value standardized with the case where the elapsed time is 0 minute as 1.

TABLE 1
Fluorescence peak (green)

| Elapsed | | | | | |
|---|---|---|---|---|---|
| time | Not | Glu | Glu | Lac | Lac |
| [min] | added | 1.0 mM | 2.6 mM | 0.9 mM | 2.2 mM |
| 0 | 1 | 1 | 1 | 1 | 1 |
| 10 | 0.98 | 1.06 | 1.04 | 0.97 | 0.98 |
| 30 | 0.98 | 1.14 | 1.16 | 0.96 | 1.02 |

TABLE 2
Fluorescence peak (red)

| Elapsed | | | | | |
|---|---|---|---|---|---|
| time | Not | Glu | Glu | Lac | Lac |
| [min] | added | 1.0 mM | 2.6 mM | 0.9 mM | 2.2 mM |
| 0 | 1 | 1 | 1 | 1 | 1 |
| 10 | 0.94 | 0.99 | 1.03 | 0.93 | 0.90 |
| 30 | 0.92 | 1.01 | 1.08 | 0.87 | 0.81 |

FIG. 7 is a graph illustrating the relationship between the glucose concentration and the lactic acid concentration, and the green fluorescence peak intensity. FIG. 8 is a graph illustrating the relationship between the glucose concentration and the lactic acid concentration, and the red fluorescence peak intensity.

From FIG. 7, the green fluorescence peak increased when glucose (Glu) was added, whereas the change was small when lactic acid (Lac) was added.

From FIG. 8, the red fluorescence peak increased when glucose (Glu) was added, but decreased when lactic acid (Lac) was added.

From the above results, when the glucose concentration is specified by the green signal and then the contribution of glucose is subtracted from the red signal, the lactic acid concentration can also be specified. Accordingly, both the glucose concentration and the lactic acid concentration can be detected using one sensor.

In the optical sensor of the present disclosure, the fluorescent unit (fluorescent layer) may contain a quantum dot for reference. In this case, the quantum dot for reference may be, for example, a core-shell quantum dot including a core and a shell covering the core, and the periphery of the quantum dot may be covered with a protective film such as glass beads. Since the quantum dot for reference is confined in a protective film such as a glass bead, the quantum dot for reference does not react with a detection target and emits constant light as a reference. Accordingly, on the basis of the light emission, for example, in view of the ratio between the red light emission and the green light emission, the sensor response can be detected without being affected by the state of the intermediate path.

In the optical sensor of the present disclosure, at least one kind of quantum dot and an enzyme may be conjugated. In this case, one kind of quantum dot and an enzyme may be conjugated, two or more kinds of quantum dot and an enzyme may be conjugated, or all kinds of quantum dot and an enzyme may be conjugated.

When the quantum dot and an enzyme are conjugated, an enzyme reaction occurs in the vicinity of the quantum dot, so that the response of the sensor is improved. As a result, the sensitivity of the sensor can be enhanced or the response time can be shortened.

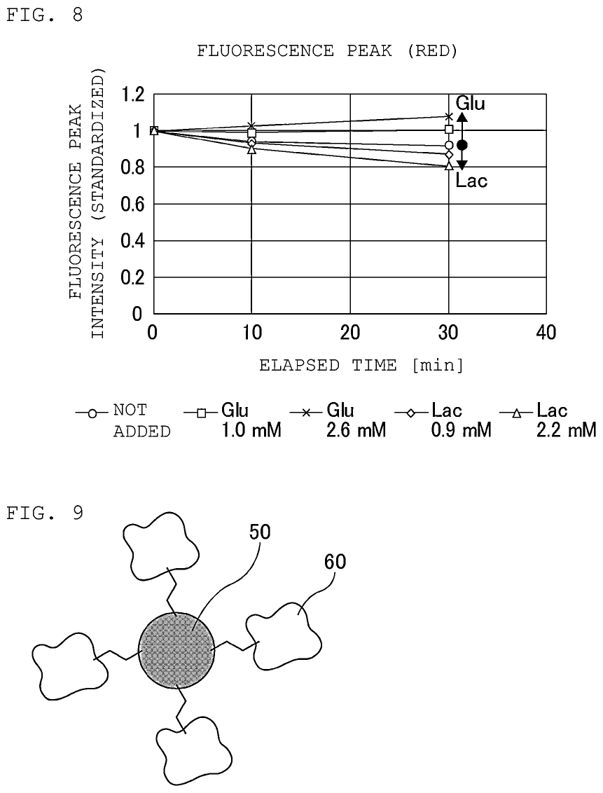

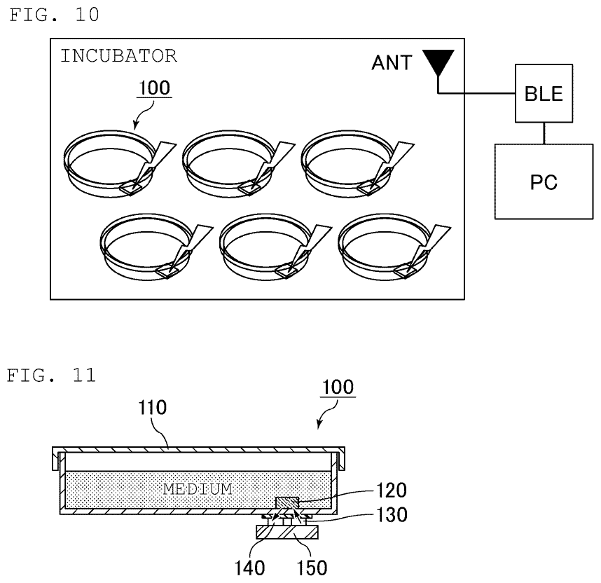

FIG. 9 is a schematic view illustrating an example of a state in which a quantum dot and enzymes are conjugated.

In the example illustrated in FIG. 9, the quantum dot 50 is conjugated with four enzymes 60.

The quantum dot 50 is, for example, a ZnS—AgInS₂ solid solution (ZAIS).

To the quantum dot 50, a ligand such as mercaptopropionic acid may be coordinated.

The enzyme 60 is, for example, glucose oxidase (GOx, GOD) or glucose dehydrogenase (GDH).

### [Detection Device]

A detection device of the present disclosure includes: a container having optical transparency; a sensor unit arranged inside the container; and a light emitting element and a light receiving element arranged outside the container so as to face the sensor unit, in which the sensor unit is the optical sensor of the present disclosure.

In the detection device of the present disclosure, data acquired from the sensor unit arranged inside the container can be read outside the container. In this way, since the acquisition and reading of data are separated inside and outside the container, contamination of bacteria and the like into the container can be prevented.

The detection device of the present disclosure can flexibly cope with various container forms. Furthermore, by arranging a large number of the optical sensors of the present disclosure in one container, multipoint measurement can be supported.

For example, by using the detection device of the present disclosure, the metabolism of cells can be monitored.

The detection device of the present disclosure preferably further includes, outside the container, a communication unit that transmits data acquired from the sensor unit to an external device. In particular, the communication unit preferably wirelessly transmits data acquired from the sensor unit to the external device.

FIG. 10 is a schematic view illustrating a detection device according to an embodiment of the present disclosure. FIG. 11 is a schematic view in which the detection device illustrated in FIG. 10 is enlarged.

The detection device 100 illustrated in FIGS. 10 and 11 is applied to a container 110 for small amount culture such as a petri dish or a flask. In the example illustrated in FIG. 10, the container 110 is arranged in an incubator.

In the detection device 100, the sensor unit 120 is arranged inside the container 110, and the light emitting element 130 and the light receiving element 140 are arranged outside the container 110 so as to face the sensor unit 120. The sensor unit 120 is, for example, the optical sensor 1 illustrated in FIG. 5.

The detection device 100 preferably further includes, outside the container 110, a communication unit 150 that transmits data acquired from the sensor unit 120 to an external device. In the example illustrated in FIG. 10, the communication unit 150 wirelessly transmits data acquired from the sensor unit 120 to a computer PC that is an example of the external device. On the side of the computer PC that is an example of the external device, for example, data conforming to a standard such as BLE (Bluetooth (registered trademark) Low Energy) are received by an antenna ANT.

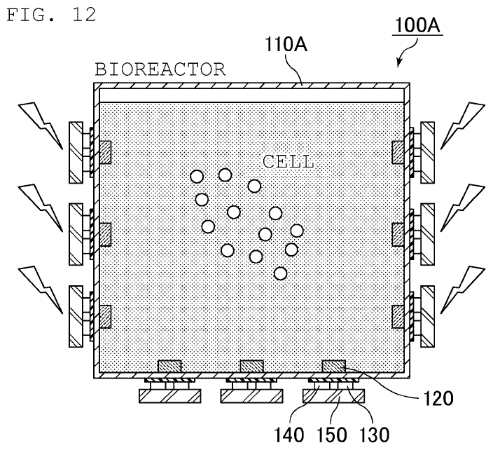

FIG. 12 is a schematic view illustrating a detection device according to another embodiment of the present disclosure.

The detection device 100A illustrated in FIG. 12 is applied to a container 110A for mass culture such as a bioreactor.

In the detection device 100A, the plurality of sensor units 120 are arranged inside the container 110A, and the light emitting elements 130 and the light receiving elements 140 are arranged outside the container 110A so as to face the respective sensor units 120. The sensor unit 120 is, for example, the optical sensor 1 illustrated in FIG. 5.

The detection device 100A preferably further includes, outside the container 110A, a communication unit 150 that transmits data acquired from the sensor unit 120 to an external device (not illustrated).

As still another embodiment of the present disclosure, the container (not shown) configuring the detection device may be a single use bag (disposable bag). In this case, the sensor unit may be attached to the single use bag or may be pressure bonded for incorporation.

The optical sensor of the present disclosure is not limited to the above embodiment as long as the optical sensor includes a fluorescent unit containing a plurality of kinds of quantum dot, enzyme, and quencher in a hydrogel. Similarly, the detection device of the present disclosure is not limited to the above embodiment as long as a sensor unit is arranged inside a container having optical transparency, a light emitting element and a light receiving element are arranged outside the container so as to face the sensor unit, and the sensor unit is the optical sensor of the present disclosure. Accordingly, various applications and modifications can be made within the scope of the present disclosure regarding the configurations, manufacturing conditions, and the like of the optical sensor and the detection device.

The following content is disclosed in the present specification.

<1> An optical sensor including a fluorescent unit containing a plurality of kinds of quantum dots, enzymes, and quenchers in a hydrogel.

<2> The optical sensor according to <1>, wherein the plurality of kinds of quantum dots, enzymes, and quenchers include: a first quencher having a first LUMO energy level combined with a first quantum dot having a first energy level at a lower end of a conduction band thereof, and a second quencher having a second LUMO energy level combined with a second quantum dot having a second energy level at a lower end of a conduction band thereof, wherein the first LUMO energy level is higher than the second LUMO energy level, and the first energy level is higher than the second energy level.

<3> The optical sensor according to <1> or <2>, wherein the plurality of kinds of quantum dots, enzymes, and quenchers include: a first quencher having a first LUMO energy level combined with a first quantum dot having a first band gap, and a second quencher having a second LUMO energy level combined with a second quantum dot having a second band gap, wherein the first LUMO energy level is lower than the second LUMO energy level, and the first band gap is lower than the second band gap.

<4> The optical sensor according to any one of <1> to <3>, further including a reference quantum dot in the hydrogel.

<5> The optical sensor according to any one of <1> to <4>, wherein the fluorescent unit includes a fluorescent layer, and the optical sensor further includes a support member having optical transparency on a first principal surface side of the fluorescent layer.

<6> The optical sensor according to <5>, further including a light shielding layer on a second principal surface side of the fluorescent layer opposite the first principal surface side of the fluorescent layer.

<7> The optical sensor according to <6>, further including a reflection layer between the fluorescent layer and the light shielding layer.

<8> The optical sensor according to any one of <1> to <7>, wherein, among the plurality of the kinds of quantum dots, enzymes, and quenchers, at least one quantum dot and enzyme are conjugated.

<9> The optical sensor according to any one of <1> to <8>, wherein the hydrogel is nonionic.

<10> A detection device including: a container having optical transparency; a sensor unit arranged inside the container; and a light emitting element and a light receiving element arranged outside the container so as to face the sensor unit, wherein the sensor unit is the optical sensor according to any one of <1> to <9>.

<11> The detection device according to <10>, further including a communication unit that transmits data acquired from the sensor unit to an external device.

### DESCRIPTION OF REFERENCE SYMBOLS

1: Optical sensor
10: Fluorescent layer (fluorescent unit)
15: Hydrogel
20: Support member
30: Light shielding layer
40: Reflection layer
50: Quantum dot
51: Red quantum dot
51A: Core of red quantum dot
51B: Shell of red quantum dot
52: Green quantum dot
52A: Core of green quantum dot
52B: Shell of green quantum dot
60: Enzyme
70: Quencher
100, 100A: Detection device
110, 110A: Container
120: Sensor unit
130: Light emitting element
140: Light receiving element
150: Communication unit

1. An optical sensor comprising: a fluorescent unit containing a plurality of kinds of quantum dots, enzymes, and quenchers in a hydrogel.

2. The optical sensor according to claim 1, wherein the plurality of kinds of quantum dots, enzymes, and quenchers include:
a first quencher having a first LUMO energy level combined with a first quantum dot having a first energy level at a lower end of a conduction band thereof, and
a second quencher having a second LUMO energy level combined with a second quantum dot having a second energy level at a lower end of a conduction band thereof,
wherein the first LUMO energy level is higher than the second LUMO energy level, and the first energy level is higher than the second energy level.

3. The optical sensor according to claim 1, wherein the plurality of kinds of quantum dots, enzymes, and quenchers include:
a first quencher having a first LUMO energy level combined with a first quantum dot having a first band gap, and
a second quencher having a second LUMO energy level combined with a second quantum dot having a second band gap,
wherein the first LUMO energy level is lower than the second LUMO energy level, and the first band gap is lower than the second band gap.

4. The optical sensor according to claim 1, further comprising a reference quantum dot in the hydrogel.

5. The optical sensor according to claim 1, wherein the fluorescent unit comprises a fluorescent layer, and the optical sensor further comprises a support member having optical transparency on a first principal surface side of the fluorescent layer.

6. The optical sensor according to claim 5, further comprising a light shielding layer on a second principal surface side of the fluorescent layer opposite the first principal surface side of the fluorescent layer.

7. The optical sensor according to claim 6, further comprising a reflection layer between the fluorescent layer and the light shielding layer.

8. The optical sensor according to claim 1, wherein, among the plurality of the kinds of quantum dots, enzymes, and quenchers, at least one quantum dot and enzyme are conjugated.

9. The optical sensor according to claim 1, wherein the hydrogel is nonionic.

10. A detection device comprising:
a container having optical transparency;
a sensor unit comprising the optical sensor according to claim 1 arranged inside the container; and
a light emitting element and a light receiving element arranged outside the container so as to face the sensor unit.

11. The detection device according to claim 10, further comprising a communication unit that transmits data acquired from the sensor unit to an external device.

12. The detection device according to claim 10, wherein the plurality of kinds of quantum dots, enzymes, and quenchers in the optical sensor include:
a first quencher having a first LUMO energy level combined with a first quantum dot having a first energy level at a lower end of a conduction band thereof, and
a second quencher having a second LUMO energy level combined with a second quantum dot having a second energy level at a lower end of a conduction band thereof,
wherein the first LUMO energy level is higher than the second LUMO energy level, and the first energy level is higher than the second energy level.

13. The detection device according to claim 10, wherein the plurality of kinds of quantum dots, enzymes, and quenchers in the optical sensor include:
a first quencher having a first LUMO energy level combined with a first quantum dot having a first band gap, and
a second quencher having a second LUMO energy level combined with a second quantum dot having a second band gap,
wherein the first LUMO energy level is lower than the second LUMO energy level, and the first band gap is lower than the second band gap.

14. The detection device according to claim 10, wherein the optical sensor further comprises a reference quantum dot in the hydrogel.

15. The detection device according to claim 10, wherein the fluorescent unit of the optical sensor comprises a fluorescent layer, and the optical sensor further comprises a support member having optical transparency on a first principal surface side of the fluorescent layer.

16. The detection device according to claim 15, wherein the optical sensor further comprises a light shielding layer on a second principal surface side of the fluorescent layer opposite the first principal surface side of the fluorescent layer.

17. The detection device according to claim 16, wherein the optical sensor further comprises a reflection layer between the fluorescent layer and the light shielding layer.

18. The detection device according to claim 10, wherein, among the plurality of the kinds of quantum dots, enzymes, and quenchers of the optical sensor, at least one quantum dot and enzyme are conjugated.

19. The detection device according to claim 10, wherein the hydrogel of the optical sensor is nonionic.
